(19) United States
(12) Patent Application Publication
(10) Pub. No.: US 2019/0175016 A1
(43) Pub. Date: Jun. 13, 2019

(54) Calculate Physiological State and Control Smart Environments via Wearable Sensing Elements

(71) Applicant: International Business Machines Corporation, Armonk, NY (US)

(72) Inventors: Anni R. Coden, Bronx, NY (US); Hani T. Jamjoom, Cos Cob, CT (US); David M. Lubensky, Brookfield, CT (US); Justin Gregory Manweiler, Somers, NY (US); Katherine Vogt, New York, NY (US); Justin Weisz, Stamford, CT (US)

(21) Appl. No.: 16/218,612

(22) Filed: Dec. 13, 2018

Related U.S. Application Data
(63) Continuation-in-part of application No. 15/837,572, filed on Dec. 11, 2017, now abandoned.

(51) Int. Cl. A61B 5/00; A61B 5/024; A61B 5/08; A61B 5/053; A61B 5/11; G08B 21/04; A61B 5/04; G06F 1/16; G06F 1/3206

(52) U.S. Cl. CPC A61B 5/0002; G06F 3/015; A61B 5/02405; A61B 5/7267; A61B 5/08; A61B 5/6801; A61B 5/0531; A61B 5/1121; G08B 21/0446; A61B 5/04; G06F 1/163; G06F 1/3206; A61B 5/1118; A61B 5/1112; G06F 1/1626; A61B 5/02438; A61B 5/7282

(57) ABSTRACT

An approach is disclosed that receives, at a wearable sensing element worn by a user, sensor data that pertains to the user's physiological functions. Physiological states pertaining to the user are calculated from the received sensor data, with the physiological states including both physical states and mental states. The calculated physiological state is matched to an environmental action states, and environmental actions are responsively performed to change a physical environment of the user.

### BACKGROUND

Wearable technology, also called “wearable sensing elements,” are smart electronic devices that can be worn on the body as implants or accessories, such as around the wrist or chest. These wearable sensing elements are electronic devices with micro-controllers that can often detect human body measurements, such as pulse or blood pressure. Wearable sensing elements such as activity trackers are a good example of the Internet of Things, since “things” such as electronics, software, sensors, and connectivity are effectors that enable objects to exchange data through the Internet with a manufacturer, operator, and/or other connected devices, such as the user's computer system or smart phone, without requiring human intervention. Wearable technology has a variety of applications which grows as the field itself expands. It appears prominently in consumer electronics with the popularization of “smartwatches” and activity trackers.

### SUMMARY

An approach is disclosed that receives, at a wearable sensing element worn by a user, sensor data that pertains to the user's physical states. Physical states pertaining to the user are calculated from the received sensor data, with the physical states including both physical state data and mental state data. The calculated physiological state is matched to an environmental action, and environmental actions are responsively performed to change a physical environment of the user.

The foregoing is a summary and thus contains, by necessity, simplifications, generalizations, and omissions of detail; consequently, those skilled in the art will appreciate that the summary is illustrative only and is not intended to be in any way limiting. Other aspects, inventive features, and advantages will become apparent in the non-limiting detailed description set forth below.

### BRIEF DESCRIPTION OF THE DRAWINGS

This disclosure may be better understood by referencing the accompanying drawings, wherein:

FIG. 1 is a block diagram of a data processing system in which the methods described herein can be implemented;

FIG. 2 provides an extension of the information handling system environment shown in FIG. 1 to illustrate that the methods described herein can be performed on a wide variety of information handling systems which operate in a networked environment;

FIG. 3 is a component diagram depicting components used in a smart environment that is partially controlled by inter-physiological state of a user as detected by a wearable sensing element;

FIG. 4 is a flowchart depicting steps taken to configure the wearable sensing element to control the user's environment;

FIG. 5 is a flowchart depicting steps taken by a process that collects sensor data and train a model that is used to control the user's smart environment; and

FIG. 6 is a flowchart depicting steps taken by a rules engine that operates to perform actions based on a trained model and the data received from the wearable sensing element sensors to control the smart environment.

### DETAILED DESCRIPTION

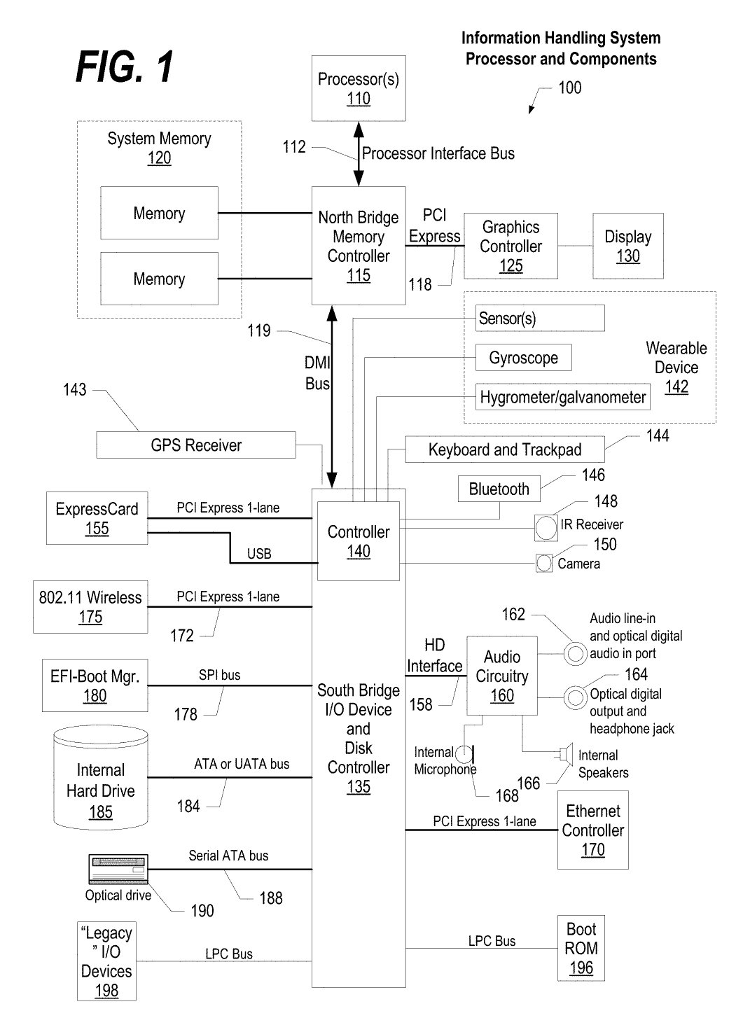

FIGS. 1-6 show an approach for changing the state of IoT (Internet of Things) devices in the physical world based on the physical and mental states of a person within an environment. A set of sensors measures biometric signals from a person and those signals are mapped to higher-order mental and physical states. For example, a heart rate monitor may be used to measure how physically active someone is; when that person is exercising (sustained increase in heart rate), this may trigger turning on the air conditioner to make the environment cooler.

A simplified set of steps used in one embodiment of the approach are as follows. First, a person wears a set of wirelessly-connected “Internet of Things” wearable sensing elements, including (but not limited to) sensors that measure heart rate, heart rate variability, temperature, respiratory rate, blood pressure, weight, ECG, pulse oxygenation, skin conductance (galvanic skin response), accelerometers, gyroscopes, compass, location, etc. Second, raw sensor data is fed into a previously-trained machine learning classifier to calculate physical and mental states. Physical states can be calculated e.g. by using accelerometer data to calculate whether a person is stationary, walking, or running; by using heart rate/heart rate variability data can be used to determine the physical activity level of a the user. In addition, some mental states can be calculated e.g. by collecting a plurality of data from sensors along with labels specified by the person. For example, the person taps a button in a smartphone app indicating they are hungry, and the last 30 seconds of sensor data are used to train a model indicating “hunger”. This model can be used in the future to predict the onset of hunger by looking for similar patterns in the sensor data. Third, given a set of physical and mental states of a person, a set of rules may be evaluated by a Rule Engine to control physical “Internet of Things” devices. For example, if “tired” is detected, the rule engine may trigger an action for turning down the lights and playing soft music to create a relaxing environment. Another example is if “flu” is detected (high temperature), the rule engine may trigger an action to alert a care provider. Another example is if “heart attack” is detected (high heart rate variability coupled with dangerously low or no pulse), a smartphone places a call to 911 and the doors of the house are unlocked to permit emergency responders to enter. The rules may follow an if-this-then-that format, as given in the examples above. In addition, rules may be calculated by correlating changes in the physical states of connected IoT devices with the sensor data collected from a person; e.g. if a person consistently turns on the lights after returning home with an elevated heart rate, we may determine this to be a rule to perform automatically in the future.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The detailed description has been presented for purposes of illustration, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

As will be appreciated by one skilled in the art, aspects may be embodied as a system, method or computer program product. Accordingly, aspects may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. As used herein, a computer readable storage medium does not include a computer readable signal medium.

Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present disclosure are described below with reference to flowchart illustrations and/or block diagrams of methods, apparatus (systems) and computer program products. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

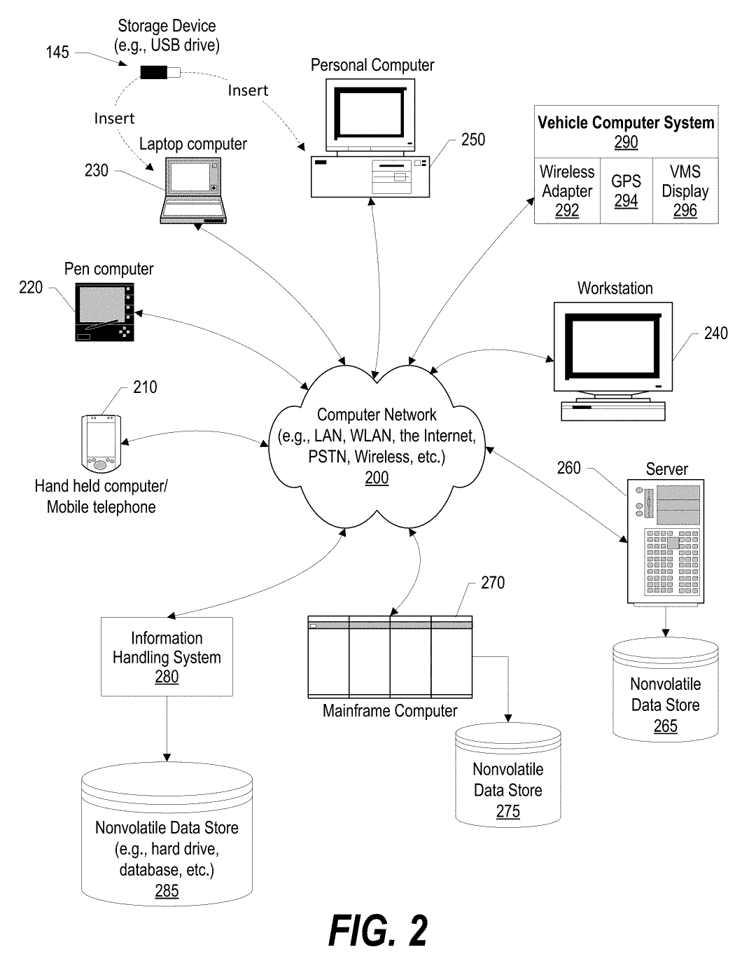

The following detailed description will generally follow the summary, as set forth above, further explaining and expanding the definitions of the various aspects and embodiments as necessary. To this end, this detailed description first sets forth a computing environment in FIG. 1 that is suitable to implement the software and/or hardware techniques associated with the disclosure. A networked environment is illustrated in FIG. 2 as an extension of the basic computing environment, to emphasize that modern computing techniques can be performed across multiple discrete devices.

FIG. 1 illustrates information handling system 100, which is a simplified example of a computer system capable of performing the computing operations described herein. Information handling system 100 includes one or more processors 110 coupled to processor interface bus 112. Processor interface bus 112 connects processors 110 to Northbridge 115, which is also known as the Memory Controller Hub (MCH). Northbridge 115 connects to system memory 120 and provides a means for processor(s) 110 to access the system memory. Graphics controller 125 also connects to Northbridge 115. In one embodiment, PCI Express bus 118 connects Northbridge 115 to graphics controller 125. Graphics controller 125 connects to display device 130, such as a computer monitor.

Northbridge 115 and Southbridge 135 connect to each other using bus 119. In one embodiment, the bus is a Direct Media Interface (DMI) bus that transfers data at high speeds in each direction between Northbridge 115 and Southbridge 135. In another embodiment, a Peripheral Component Interconnect (PCI) bus connects the Northbridge and the Southbridge. Southbridge 135, also known as the I/O Controller Hub (ICH) is a chip that generally implements capabilities that operate at slower speeds than the capabilities provided by the Northbridge. Southbridge 135 typically provides various busses used to connect various components. These busses include, for example, PCI and PCI Express busses, an ISA bus, a System Management Bus (SMBus or SMB), and/or a Low Pin Count (LPC) bus. The LPC bus often connects low-bandwidth devices, such as boot ROM 196 and “legacy” I/O devices (using a “super I/O” chip). The “legacy” I/O devices (198) can include, for example, serial and parallel ports, keyboard, mouse, and/or a floppy disk controller. The LPC bus also connects Southbridge 135 to Trusted Platform Module (TPM) 195. Other components often included in Southbridge 135 include a Direct Memory Access (DMA) controller, a Programmable Interrupt Controller (PIC), and a storage device controller, which connects Southbridge 135 to nonvolatile storage device 185, such as a hard disk drive, using bus 184.

ExpressCard 155 is a slot that connects hot-pluggable devices to the information handling system. ExpressCard 155 supports both PCI Express and USB connectivity as it connects to Southbridge 135 using both the Universal Serial Bus (USB) the PCI Express bus. Southbridge 135 includes Controller 140 that provides connectivity, such as USB connectivity, to devices that connect to the USB. These devices include webcam (camera) 150, infrared (IR) receiver 148, keyboard and trackpad 144, and Bluetooth device 146, which provides for wireless personal area networks (PANs). Controller 140 also provides USB connectivity to other wearable sensing element components 142, such as a sensors used to detect body conditions (e.g., the user's pulse, etc.), a gyroscope used to detect movement of the user, and Hygrometer/galvanometer used to detect perspiration of the user.

Wireless Local Area Network (LAN) device 175 connects to Southbridge 135 via the PCI or PCI Express bus 172. LAN device 175 typically implements one of the IEEE 802.11 standards of over-the-air modulation techniques that all use the same protocol to wireless communicate between information handling system 100 and another computer system or device. Optical storage device 190 connects to Southbridge 135 using Serial ATA (SATA) bus 188. Serial ATA adapters and devices communicate over a high-speed serial link. The Serial ATA bus also connects Southbridge 135 to other forms of storage devices, such as hard disk drives. Audio circuitry 160, such as a sound card, connects to Southbridge 135 via bus 158. Audio circuitry 160 also provides functionality such as audio line-in and optical digital audio in port 162, optical digital output and headphone jack 164, internal speakers 166, and internal microphone 168. Ethernet controller 170 connects to Southbridge 135 using a bus, such as the PCI or PCI Express bus. Ethernet controller 170 connects information handling system 100 to a computer network, such as a Local Area Network (LAN), the Internet, and other public and private computer networks.

While FIG. 1 shows one information handling system, an information handling system may take many forms. For example, an information handling system may take the form of a desktop, server, portable, laptop, notebook, or other form factor computer or data processing system. In addition, an information handling system may take other form factors such as a personal digital assistant (PDA), a gaming device, ATM machine, a portable telephone device, a communication device or other devices that include a processor and memory.

The Trusted Platform Module (TPM 195) shown in FIG. 1 and described herein to provide security functions is but one example of a hardware security module (HSM). Therefore, the TPM described and claimed herein includes any type of HSM including, but not limited to, hardware security devices that conform to the Trusted Computing Groups (TCG) standard, and entitled “Trusted Platform Module (TPM) Specification Version 1.2.” The TPM is a hardware security subsystem that may be incorporated into any number of information handling systems, such as those outlined in FIG. 2.

FIG. 2 provides an extension of the information handling system environment shown in FIG. 1 to illustrate that the methods described herein can be performed on a wide variety of information handling systems that operate in a networked environment. Types of information handling systems range from small handheld devices, such as handheld computer/mobile telephone 210 to large mainframe systems, such as mainframe computer 270. Examples of handheld computer 210 include personal digital assistants (PDAs), personal entertainment devices, such as MP3 players, portable televisions, and compact disc players. Other examples of information handling systems include pen, or tablet, computer 220, laptop, or notebook, computer 230, workstation 240, personal computer system 250, and server 260. Other types of information handling systems that are not individually shown in FIG. 2 are represented by information handling system 280. As shown, the various information handling systems can be networked together using computer network 200. Types of computer network that can be used to interconnect the various information handling systems include Local Area Networks (LANs), Wireless Local Area Networks (WLANs), the Internet, the Public Switched Telephone Network (PSTN), other wireless networks, and any other network topology that can be used to interconnect the information handling systems. Many of the information handling systems include nonvolatile data stores, such as hard drives and/or nonvolatile memory. Some of the information handling systems shown in FIG. 2 depicts separate nonvolatile data stores (server 260 utilizes nonvolatile data store 265, mainframe computer 270 utilizes nonvolatile data store 275, and information handling system 280 utilizes nonvolatile data store 285). The nonvolatile data store can be a component that is external to the various information handling systems or can be internal to one of the information handling systems. In addition, removable nonvolatile storage device 145 can be shared among two or more information handling systems using various techniques, such as connecting the removable nonvolatile storage device 145 to a USB port or other connector of the information handling systems.

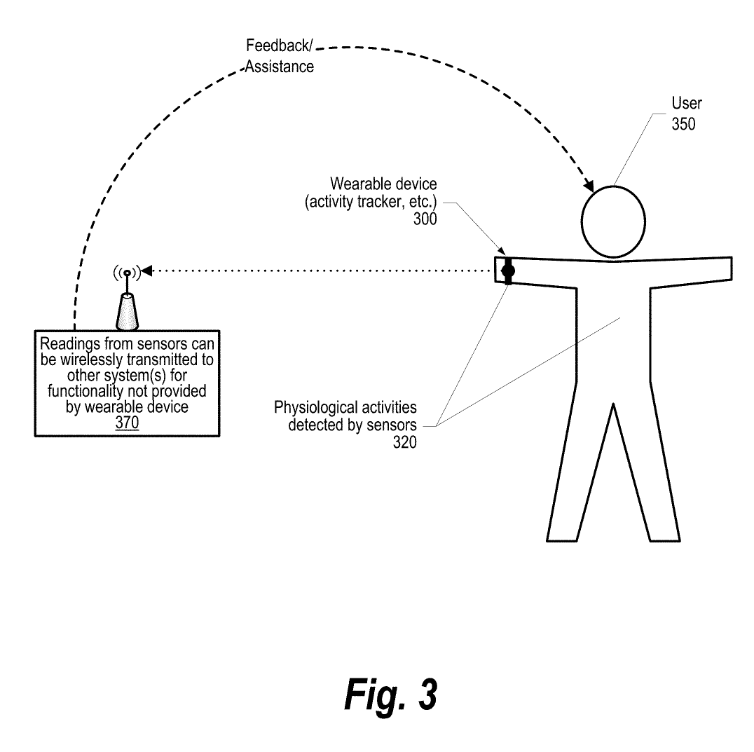

FIG. 3 is a component diagram depicting components used in a smart environment that is partially controlled by inter-physiological state of a user as detected by a wearable sensing element. Wearable sensing element 300 is worn by user 350. The wearable sensing element receives a set of sensor data corresponding to a current set of user physiological data pertaining to user 350. The system calculates, from the received set of sensor data, one or more physical states that pertain to the user. At least one of the physiological states is a physical state and at least one of the physiological states is a mental state. The calculated mental state of the user might be depressed, sad, happy, content, etc. and the calculated physical state might be tired, energetic, asleep, etc.

In one embodiment, the received set of sensor data is transmitted to a second device, such as external device 370, so that the second device can perform actions or other functionality not provided by the wearable sensing element. The approach then matches the user's calculated physiological states a set of environmental action states with the environmental state actions corresponding to an environmental action that is performed when a match occurs. Examples of environmental actions might be to a change an ambient light level, change a temperature setting, change a sound level setting, change a fan speed level, or change a humidity level setting. In one embodiment, the system can be configured so that, for example, if the system determines that the user is physically tired but mentally happy, then the system might automatically perform environmental actions such as dim the ambient light level as well as play soft ambient music to the user in the environment. In one embodiment, a neural network model is trained based on the sensor readings and the users physiological states so that, over time, the system is better able to calculate the user's physiological and mental states based on the current readings from the sensors.

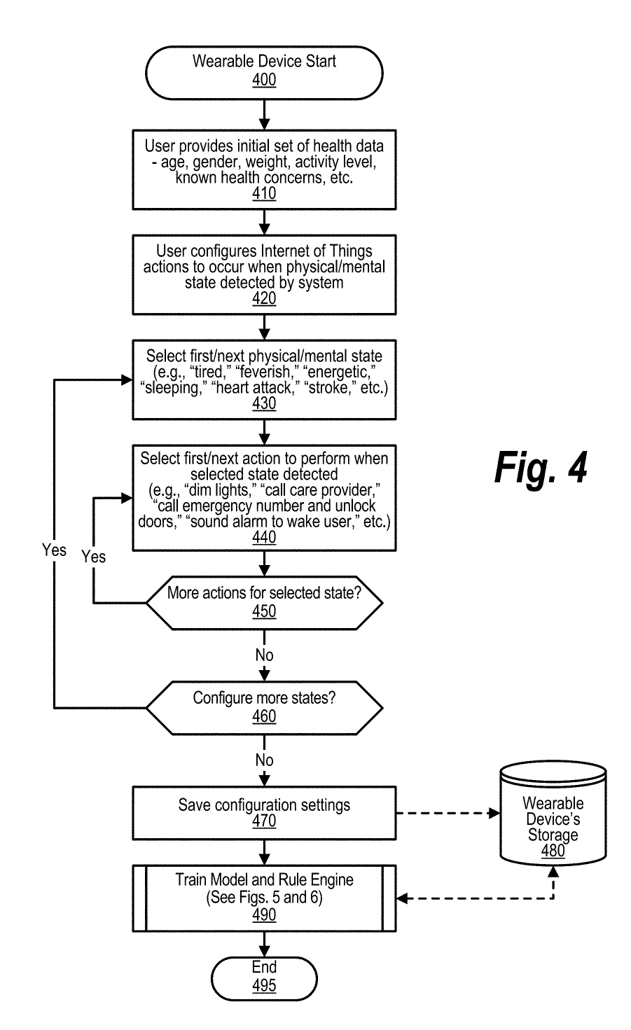

FIG. 4 is a flowchart depicting steps taken to configure the wearable sensing element to control the user's environment. FIG. 4 processing commences at 400 and shows the steps taken by a process that configures environmental actions that automatically are performed based on readings received from a wearable sensing element. At step 410, the process provides initial set of physical and physiological data pertaining to the user. This data might include the user's age, gender, weight, height, activity level, as well as any known health concerns or issues of the user, etc. At step 420, the process configures the Internet of Things actions that should occur based on the user's physiological states that include both the user's physical and mental states.

At step 430, the process selects the first physical or mental state, such as the user being found to be “tired,” “feverish,” “energetic,” “sleeping,” suffering a “heart attack,” “stroke,” etc. At step 440, the process selects the first environmental action that should be performed when the selected physical or mental state is detected. For example, the environmental action could be to dim the ambient lighting, call a care provider, call emergency number and unlock doors, sound alarm to wake the user, change the temperature in the environment, change sound settings (e.g., ambient music, volume level, etc.), change air flow settings such as by changing a fan speed level, changing a humidity level by increasing the humidity level with a humidifier or decreasing the humidity level with a dehumidifier, etc. The process next determines whether the user wants more environmental actions to occur for the selected physiological state (decision 450). If the user wants more actions to occur, then decision 450 branches to the ‘yes’ branch which loops back to step 440 to select the next environmental action. This looping continues until the user does not want any more actions to occur, at which point decision 450 branches to the ‘no’ branch exiting the loop. The process then determines whether the user wants to configure environmental actions to perform for additional physiological and mental states (decision 460). If the user wants to configure actions for additional physiological and mental states, then decision 460 branches to the ‘yes’ branch which loops back to step 430 to select and process the next physiological or mental state as described above. This looping continues until the user is finished configuring physiological and mental states, at which point decision 460 branches to the ‘no’ branch exiting the loop.

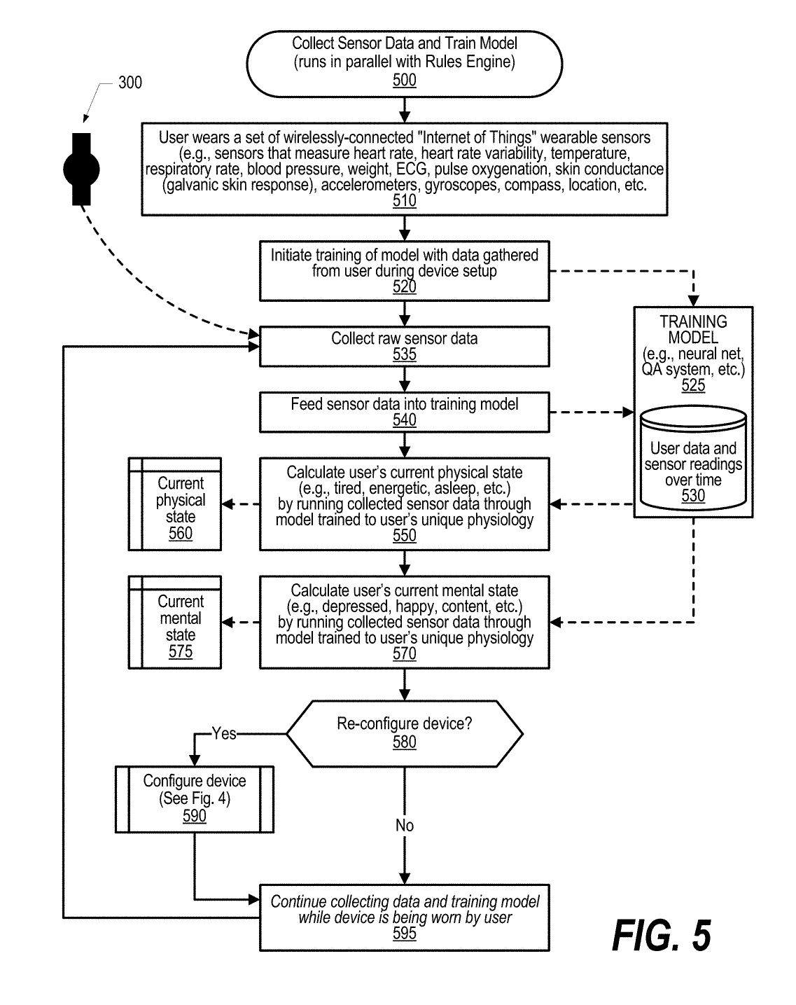

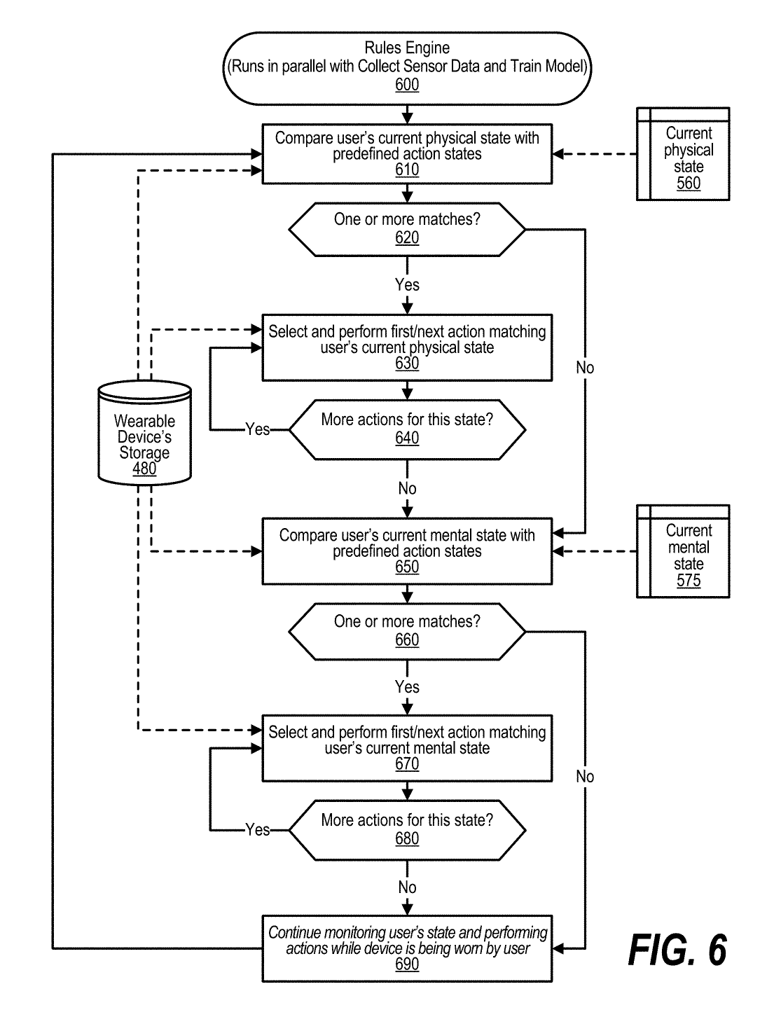

At step 470, the process saves the configuration settings in data store 480. At predefined process 490, the process performs the Train Neural Network Model and Rule Engine routine (see FIGS. 5 and 6 and corresponding text for processing details). FIG. 4 processing thereafter ends at 495.

FIG. 5 is a flowchart depicting steps taken by a process that collects sensor data from a wearable sensing element and trains a model that is used to control the user's smart environment. FIG. 5 processing commences at 500 and shows the steps taken by a process that collects sensor data from a wearable sensing element and also trains a neural network model used to calculate the user's physical and mental states. This routine executes in parallel with the Rules Engine process that is shown in FIG. 6. At step 510, the users wears a set of one or more wirelessly-connected “Internet of Things” wearable sensing elements, such as those included in wearable sensing element 300. These sensors might be able to measure the user's heart rate, the user's heart rate variability, the user's body temperature, the user's respiratory rate, the user's blood pressure, weight, ECG, pulse oxygenation, skin conductance (galvanic skin response), and activity level via sensors such as accelerometer, gyroscopes, compass, GPS location, and the like.

At step 520, the process initiates the training of model 525 with data gathered at the wearable sensing element and also data gathered from the user while the user was configuring the wearable sensing element, as shown in FIG. 4. At step 535, the process collects raw sensor data from wearable sensing element 300. At step 540, the process feeds the collected sensor data into training model 525. Training model 525 retains a history of user sensor readings collected over time as well as any user data that was gathered during setup processing. This data is stored in data store 530. At step 550, the process calculates the user's current physical state (e.g., tired, energetic, asleep, etc.) based on the current collected sensor data and the model that has been trained to the user's unique physiology. The user's current physical state that is calculated is stored in memory area 560. At step 570, the process calculates the user's current mental state (e.g., depressed, happy, content, etc.) with the calculation being based on the current collected sensor data and the model that has been trained to the user's unique physiology. The user's current mental state that is calculated is stored in memory area 575.

The process determines whether the user has requested to re-configure (or perform additional configuration) of the wearable sensing element (decision 580). If the user has requested to reconfiguring, then decision 580 branches to the ‘yes’ branch whereupon, at predefined process 590, the process performs the configure wearable sensing element routine (see FIG. 4 and corresponding text for details). For example, perhaps the user arrived home in a depressed state and noticed that the user's home was too dark and warm. The user could reconfigure the actions performed to set the ambient light level to a more suitable level and could also have the temperature of the home automatically adjusted to a more appropriate level when the system notices that the user is in a depressed state. On the other hand, if the user has not requested to reconfigure the device, then decision 580 branches to the ‘no’ branch bypassing predefined process 590.

At step 595, the process continues collecting sensor data from the wearable sensing element and continues training model 525 while the wearable sensing element is being worn by user. Processing repeatedly loops back to step 535 to process additional sensor data that is received at the wearable sensing element.

FIG. 6 is a flowchart depicting steps taken by a rules engine that operates to perform actions based on a trained model and the data received from the wearable sensing element sensors to control the smart environment. FIG. 6 processing commences at 600 and shows the steps taken by a Rules Engine process. The Rules Engine runs in parallel with the Collect Sensor Data and Train Model process shown in FIG. 5 and the Rules Engine uses the user's current physical and mental states that was calculated by the processing shown in FIG. 5. At step 610, the process compares the user's current physical state that was calculated by the processing shown in FIG. 5 and stored in memory area 560 with the predefined environmental action states that were configured using the process shown in in FIG. 4 and stored in data store 480. The process determines as to whether the comparison at step 610 resulted in any matches (decision 620). If one or more matches were found, then decision 620 branches to the ‘yes’ branch to perform steps 630 and 640. On the other hand, if there are no matches, then decision 620 branches to the ‘no’ branch bypassing steps 630 and 640. If matches were found with the user's current physical state and one or more environmental action states, then, at step 630, the process selects and performs the first environmental action matching the user's current physical state with the environmental actions being retrieved from data store 480. The process determines as to whether there are more environmental actions to perform for this physical state (decision 640). If there are more environmental actions to perform for this physical state, then decision 640 branches to the ‘yes’ branch which loops back to step 630 to select and perform the next environmental action matching the user's current physical state. This looping continues until no more environmental actions are to be performed for the user's current physical state, at which point decision 640 branches to the ‘no’ branch exiting the loop.

At step 650, the process compares the user's current mental state that was calculated by the processing shown in FIG. 5 and stored in memory area 575 with the predefined environmental action states that were configured using the process shown in in FIG. 4 and stored in data store 480. The process determines as to whether the comparison at step 650 resulted in any matches (decision 660). If one or more matches were found, then decision 660 branches to the ‘yes’ branch to perform steps 670 and 680. On the other hand, if there are no matches, then decision 660 branches to the ‘no’ branch bypassing steps 670 and 680.

If matches were found with the user's current mental state and one or more environmental action states, then, at step 670, the process selects and performs the first environmental action matching the user's current mental state with the environmental actions being retrieved from data store 480. The process determines as to whether there are more environmental actions to perform for this mental state (decision 680). If there are more environmental actions to perform for this mental state, then decision 680 branches to the ‘yes’ branch which loops back to step 670 to select and perform the next environmental action matching the user's current mental state. This looping continues until no more environmental actions are to be performed for the user's current mental state, at which point decision 680 branches to the ‘no’ branch exiting the loop.

At step 690, the process continues monitoring user's physical and mental states as found by the processing shown in FIG. 5 and, when these states change, the process loops back to step 610 to re-performing the processing described above.

While particular embodiments have been shown and described, it will be obvious to those skilled in the art that, based upon the teachings herein, that changes and modifications may be made without departing from this invention and its broader aspects. Therefore, the appended claims are to encompass within their scope all such changes and modifications as are within the true spirit and scope of this invention. Furthermore, it is to be understood that the invention is solely defined by the appended claims. It will be understood by those with skill in the art that if a specific number of an introduced claim element is intended, such intent will be explicitly recited in the claim, and in the absence of such recitation no such limitation is present. For non-limiting example, as an aid to understanding, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to inventions containing only one such element, even when the same claim includes the introductory phrases “one or more” or “at least one” and indefinite articles such as “a” or “an”; the same holds true for the use in the claims of definite articles.

What is claimed is:

1. A method, implemented by an information handling system comprising a processor and a memory accessible by the processor, the method comprising:
receiving, at a wearable sensing element worn by a user, a set of sensor data corresponding to a current set of user physiological functions pertaining to the user;
calculating, from the received set of sensor data, a plurality of physiological states pertaining to the user, wherein at least one of the physiological states is a physical state and wherein at least one of the physiological states is a mental state;
matching the calculated physiological states to a plurality of environmental action states; and
performing one or more environmental actions to change a physical environment of the user, wherein the performed environmental actions correspond to one or more of the plurality of environmental states that matched the calculated physiological states.

2. The method of claim 1 further comprising:
training a neural network model with a plurality of collections of sensor data received at the wearable sensing element over a period of time, the method further comprising:
inputting the received sensor data to the trained neural network model; and
receiving, from the trained neural network model, the calculated plurality of physiological states.

3. The method of claim 1 wherein at least one of the performed environmental actions is selected from the group consisting of a change to an ambient light level of the physical environment, a change to a temperature of the physical environment, a change to a sound level of a sound system in the physical environment, a change to a fan speed level of a fan in the physical environment, and a change to a humidity level of the physical environment.

4. The method of claim 1 further comprising:
transmitting the received set of sensor data to a second device, wherein the second device performs the environmental actions.

5. The method of claim 1 further comprising:
prior to the performance of the environmental actions:
configuring a plurality of environmental actions that include the one or more environmental actions, wherein the configuring includes associating each of the configured environmental actions with one or more environmental states, wherein the environmental states are included in the plurality of physiological states; and
storing the configured environmental actions in a data store, wherein the matching retrieves the environmental states from the data store and results in the configured environmental actions that match the user's calculated physiological states.

6. The method of claim 1 wherein at least one of the mental states is selected from the group consisting of a depressed mental state, a sad mental state, a happy mental state, and a content mental state.

7. The method of claim 1 wherein at least one of the physical states is selected from the group consisting of a tired physical state, an energetic physical state, and an asleep physical state.

8. A wearable information handling system comprising:
one or more processors;
a memory coupled to at least one of the processors; and
a set of instructions stored in the memory and executed by at least one of the processors to:
receiving, at a wearable sensing element worn by a user, a set of sensor data corresponding to a current set of user physiological functions pertaining to the user;
calculating, from the received set of sensor data, a plurality of physiological states pertaining to the user, wherein at least one of the physiological states is a physical state and wherein at least one of the physiological states is a mental state;
matching the calculated physiological states to a plurality of environmental action states; and
performing one or more environmental actions to change a physical environment of the user, wherein the performed environmental actions correspond to one or more of the plurality of environmental states that matched the calculated physiological states.

9. The information handling system of claim 8 wherein the actions further comprise:
training a neural network model with a plurality of collections of sensor data received at the wearable sensing element over a period of time, the
inputting the received sensor data to the trained neural network model; and
receiving, from the trained neural network model, the calculated plurality of physiological states.

10. The information handling system of claim 8 wherein at least one of the performed environmental actions is selected from the group consisting of a change to an ambient light level of the physical environment, a change to a temperature of the physical environment, a change to a sound level of a sound system in the physical environment, a change to a fan speed level of a fan in the physical environment, and a change to a humidity level of the physical environment.

11. The information handling system of claim 8 wherein the actions further comprise:
transmitting the received set of sensor data to a second device, wherein the second device performs the environmental actions.

12. The information handling system of claim 8 wherein the actions further comprise:
prior to the performance of the environmental actions:
configuring a plurality of environmental actions that include the one or more environmental actions, wherein the configuring includes associating each of the configured environmental actions with one or more environmental states, wherein the environmental states are included in the plurality of physiological states; and
storing the configured environmental actions in a data store, data store and results in the configured environmental actions that match the user's calculated physiological states.

13. The information handling system of claim 8 wherein at least one of the mental states is selected from the group consisting of a depressed mental state, a sad mental state, a happy mental state, and a content mental state.

14. The information handling system of claim 8 wherein at least one of the physical states is selected from the group consisting of a tired physical state, an energetic physical state, and an asleep physical state.

15. A computer program product comprising:
a computer readable storage medium comprising a set of computer instructions, the computer instructions effective to:
receiving, at a wearable sensing element worn by a user, a set of sensor data corresponding to a current set of user physiological functions pertaining to the user;
calculating, from the received set of sensor data, a plurality of physiological states pertaining to the user, wherein at least one of the physiological states is a physical state and wherein at least one of the physiological states is a mental state;
matching the calculated physiological states to a plurality of environmental action states; and
performing one or more environmental actions to change a physical environment of the user, wherein the performed environmental actions correspond to one or more of the plurality of environmental states that matched the calculated physiological states.

16. The computer program product of claim 15 wherein the actions further comprise:
training a neural network model with a plurality of collections of sensor data received at the wearable sensing element over a period of time, the computer program product wherein the actions further comprise:
inputting the received sensor data to the trained neural network model; and
receiving, from the trained neural network model, the calculated plurality of physiological states.

17. The computer program product of claim 15 wherein at least one of the performed environmental actions is selected from the group consisting of a change to an ambient light level of the physical environment, a change to a temperature of the physical environment, a change to a sound level of a sound system in the physical environment, a change to a fan speed level of a fan in the physical environment, and a change to a humidity level of the physical environment.

18. The computer program product of claim 15 wherein the actions further comprise:
transmitting the received set of sensor data to a second device, wherein the second device performs the environmental actions.

19. The computer program product of claim 15 wherein the actions further comprise:
prior to the performance of the environmental actions:
configuring a plurality of environmental actions that include the one associating each of the configured environmental actions with one or more environmental states, wherein the environmental states are included in the plurality of physiological states; and
storing the configured environmental actions in a data store, wherein the matching retrieves the environmental states from the data store and results in the configured environmental actions that match the user's calculated physiological states.

20. The computer program product of claim 15 wherein at least one of the mental states is selected from the group consisting of a depressed mental state, a sad mental state, a happy mental state, and a content mental state and wherein at least one of the physical states is selected from the group consisting of a tired physical state, an energetic physical state, and an asleep physical state.
